(19) United States
(12) Patent Application Publication
(10) Pub. No.: US 2006/0246124 A1
(43) Pub. Date: Nov. 2, 2006

(54) Methods of treating cancer with lipid-based platinum compound formulations administered intraperitoneally

(76) Inventors: Frank G. Pilkiewicz, Princeton, NJ (US); Roman Perez-Soler, New York, NY (US); Yiyu Zou, Bronx, NY (US); Kristen M. Pilkiewicz, (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/269,163

(22) Filed: Nov. 8, 2005

Related U.S. Application Data
(60) Provisional application No. 60/626,029, filed on Nov. 8, 2004.
(60) Provisional application No. 60/671,593, filed on Apr. 15, 2005.

(51) Int. Cl. A61K 33/24; A61K 31/282; A61K 9/127

(52) U.S. Cl. 424/450; 424/649; 514/492

(57) ABSTRACT

The present invention relates to a method of treating cancer in a patient comprising administering to the patient intraperitoneally, a cancer treating effective amount of a lipid-based platinum compound formulation.

### RELATED APPLICATIONS

This application claims the benefit of priority to U.S. Provisional Patent Application Ser. No. 60/626,029, filed Nov. 8, 2004, and U.S. Provisional Patent Application Ser. No. 60/671,593, filed Apr. 15, 2005.

### INTRODUCTION

Parenteral routes of administration involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is preferred over oral ones in many occurrences. For example, when the drug to be administered would partially or totally degrade in the gastrointestinal tract, parenteral administration is preferred. Similarly, where there is need for rapid response in emergency cases, parenteral administration is usually preferred over oral.

Regional delivery of chemotherapy into the peritoneal space via ip administration has been found to be a safe and effective treatment for locally recurrent cancers such as, for example, ovarian and colon cancers.

The concept of the intraperitoneal administration of antineoplastic agents in the management of cancers such as ovarian cancer has attracted the interest of numerous investigators. In fact, alkylating agents, the first cytotoxic drugs to be introduced into clinical practice, were initially examined for intraperitoneal delivery in the early 1950s. Markman M., Cancer Treat Rev., 1986, 13, 219-242.

However, it was not until the late 1970s that both the problems and potential of regional drug administration in the treatment of ovarian cancer began to be thoroughly explored. Markman M., Cancer Treat Rev., 1986, 13, 219-242; Markman M., Semin. Oncol., 1991, 18(suppl 3), 248-254. An important event in the development of a rational strategy for the examination of intraperitoneal drug delivery was the publication of a now-classic paper by Dedrick et al., from the National Cancer Institute where, for the first time, a sound pharmacokinetic rationale for this approach in the management of ovarian cancer was presented. Dedrick R L, Myers C E, Bungay P M et al., Cancer Treat. Rep., 1978, 62, 1-9.

Cisplatin—cis-diamine-dichloroplatinum (II)—is one of the more effective anti-tumor agents used in the systemic treatment of cancers. This chemotherapeutic drug is highly effective in the treatment of tumor models in laboratory animals and in human tumors, such as endometrial, bladder, ovarian and testicular neoplasms, as well as squamous cell carcinoma of the head and neck (Sur, et al., 1983 Oncology 40(5): 372-376; Steerenberg, et al., 1988 Cancer Chemother Pharmacol. 21(4): 299-307). Cisplatin is also used extensively in the treatment of lung carcinoma, both SCLC and NSCLC (Schiller et al., 2001 Oncology 61(Suppl 1): 3-13). Other active platinum compounds (defined below) are useful in cancer treatment.

Like other cancer chemotherapeutic agents, active platinum compounds such as cisplatin are typically highly toxic. The main disadvantages of cisplatin are its extreme nephrotoxicity, which is the main dose-limiting factor, its rapid excretion via the kidneys, with a circulation half life of only a few minutes, and its strong affinity to plasma proteins (Freise, et al., 1982 Arch Int Pharmacodyn Ther. 258(2): 180-192).

Attempts to minimize the toxicity of active platinum compounds have included combination chemotherapy, synthesis of analogues (Prestayko et al., 1979 Cancer Treat Rev. 6(1): 17-39; Weiss, et al., 1993 Drugs. 46(3): 360-377), immunotherapy and entrapment in liposomes (Sur, et al., 1983; Weiss, et al., 1993). Antineoplastic agents, including cisplatin, entrapped in liposomes have a reduced toxicity, relative to the agent in free form, while retaining antitumor activity (Steerenberg, et al., 1987; Weiss, et al., 1993).

Cisplatin, however, is difficult to efficiently entrap in liposomes or lipid complexes because of the bioactive agent's low aqueous solubility, approximately 1.0 mg/ml at room temperature, and low lipophilicity, both of which properties contribute to a low bioactive agent/lipid ratio.

Liposomes and lipid complexes containing cisplatin suffer from another problem—stability of the composition. In particular, maintenance of bioactive agent potency and retention of the bioactive agent in the liposome during storage are recognized problems (Freise, et al., 1982; Gondal, et al., 1993; Potkul, et al., 1991 Am J Obstet Gynecol. 164(2): 652-658; Steerenberg, et al., 1988; Weiss, et al., 1993) and a limited shelf life of liposomes containing cisplatin, on the order of several weeks at 4° C., has been reported (Gondal, et al., 1993 Eur J Cancer. 29A(11): 1536-1542; Potkul, et al., 1991).

Alberts et al. have shown that as compared with iv cisplatin, ip cisplatin significantly improves survival and has significantly fewer toxic effects in patients with stage III ovarian cancer and residual tumor masses of 2 cm or less. Alberts D. S. et al., New England Journal of Medicine, 1996, 335(26), 1950-5. However, ip cisplatin has several disadvantages such as no improvement in nephrotoxicity which is the dose-limiting toxicity.

Additionally, both preclinical and clinical data have firmly established that any benefits associated with employing the intraperitoneal route of drug delivery in the treatment of ovarian cancer are limited to a relatively well-defined small subset of patients with this malignancy. Markman M., Cancer Treat Rev., 1986, 13, 219-242; Markman M., Semin. Oncol., 1991, 18(suppl 3), 248-254; Markman M, Reichman B, Hakes T et al., J. Clin. Oncol., 1991, 9, 1801-1805. For example, in a series of patients treated at the Memorial Sloan-Kettering Cancer Center (MSKCC) with combination cisplatin-based therapy as salvage treatment of advanced ovarian cancer, 32% (17/50) of individuals whose largest residual tumor mass measured ≦1 cm in maximum diameter at the initiation of ip therapy achieved a surgically documented complete response, compared to only 5% (2/39) of patients with at least one tumor mass >1 cm in maximum diameter. Markman M, Reichman B, Hakes T et al., J. Clin. Oncol., 1991, 9, 1801-1805. Clearly more is needed than just direct routes of administration to overcome the increasingly deleterious effects of cancer.

In addition to cisplatin, a number of other antineoplastic agents have been examined for safety and potential efficacy when delivered by the ip route as salvage treatment of ovarian cancer. These include carboplatin, paclitaxel, mitoxantrone, doxorubicin, mitomycin-C, 5-fluorouracil, methotrexate, thiotepa, recombinant interferon-α, recombinant interferon-γ, interleukin 2 and tumor necrosis factor. Markman M., Cancer Treat Rev., 1986, 13, 219-242; Markman M., Semin. Oncol., 1991, 18(suppl 3), 248-254; Markman M, Reichman B, Hakes T et al., J. Clin. Oncol., 1991, 9, 1801-1805; Markman M., Regional antineoplastic drug delivery in the management of malignant disease. Baltimore: The Johns Hopkins University Press, 1991; Berek J. S., Markman M., Int. J. Gynecol. Cancer, 1992, 1, 26-29; Markman M, Berek J. S., Int. J. Gynecol. Cancer, 1992, 1, 30-34; Alberts D. S., Liu P. Y., Hannigan E. V. et al., Proc. Am. Soc. Clin. Oncol., 1995, 14, 273a; Rowinsky E. K., Donehower R. C., N. Engl. J. Med., 1995, 332, 1004-1014. Combination regimens have also been explored.

Despite the advances made with ip administration of platinum compounds, the dose limiting toxicity and low drug level in targeted tissues of platinum compounds make most therapies fail to improve patients' life-expectancy.

### SUMMARY OF THE INVENTION

It is an object of the present invention to provide a method of treating cancer comprising administering platinum compounds as part of a lipid-based formulation with lower sub-acute toxicity, in some cases by as much as two times, than when the platinum compound is administered without the lipid formulation.

It is also an object of the present invention to treat cancer by introducing platinum compounds regionally to bypass gastrointestinal degradation that is often associated with oral administration.

The subject invention results from the realization that lipid-based platinum formulations presented herein can be effectively administered intraperitoneally.

In one embodiment, the present invention features methods of treating cancer in a patient comprising intraperitoneally administering a cancer treating effective amount of a lipid-based platinum formulation to the patient. In certain embodiments, the platinum compound in the platinum formulation is administered intraperitoneally at a concentration of about 0.8 mg/ml to about 1.2 mg/ml. In certain embodiments, the platinum compound in the platinum formulation is administered intraperitoneally at a concentration of about 0.9 mg/ml to about 1.1 mg/ml. In certain embodiments, the platinum compound in the platinum formulation is administered intraperitoneally at a concentration of 1 mg/ml.

In certain embodiments the present invention relates to the aforementioned method, wherein the platinum compound is selected from the group consisting of: cisplatin, carboplatin (diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans,trans,trans)bis-mu-(hexane-1,6-diamine)-mu-[diamine-platinum(II)]bis[diamine(chloro)platinum(II)]tetrachloride, and mixture thereof. In certain embodiments the platinum compound is cisplatin.

In certain embodiments the present invention relates to the aforementioned method, wherein the lipid is comprised of a member selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic aicd (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic aicd (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In certain embodiments the lipid in the lipid-based platinum formulation is a phospholipid such as dipalmitoylphosphatidylcholine (DPPC) or a sterol, such as cholesterol, or both. In a further embodiment, the lipid is a mixture of DPPC from 50 to 65 mol % and cholesterol from 35 to 50 mol %.

In a further embodiment, the cancer treated is selected from the following: melanoma, testis (germ cell), osteosarcoma, soft tissue sarcoma, thyroid cancer, colon cancer, ovarian cancer, cancer of the kidney, breast cancer, colorectal cancer, prostate cancer, bladder cancer, uterine cancer, lung cancer, stomach cancer, liver cancer, endometrial, or squamous cell carcinomas of the head and neck. In certain embodiments, the cancer treated is ovarian or colon cancer.

In a further embodiment, the present invention relates to the aforementioned methods, wherein the ratio of platinum compound to lipid in the lipid-based platinum compound formulation is between 1:5 by weight and 1:50 by weight. In a further embodiment, the lipid-based platinum compound formulation comprises liposomes having a mean diameter of 0.01 microns to 3.0 microns.

In a further embodiment, the present invention relates to the aforementioned method, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns. In a further embodiment, the platinum compound is cisplatin.

In a further embodiment, the present invention relates to the aforementioned method, wherein the lipid is a mixture of DPPC and cholesterol in a 2 to 1 ratio by weight, the ratio of platinum compound to lipid in the lipid-based platinum compound formulation is 1:20 by weight, the lipid-based platinum compound formulation comprises liposomes having a mean diameter of 0.40 microns, and wherein the platinum compound is cisplatin.

In a further embodiment, the patient is a human. In a further embodiment, the lipid-based platinum compound formulation is administered to the patient at least once every three weeks. In a further embodiment, the lipid-based platinum compound formulation is administered to the patient at least twice every three weeks. In a further embodiment, the lipid-based platinum compound formulation is administered to the patient at least three times every three weeks. In a further embodiment, the amount of platinum compound in the lipid-based platinum compound formulation is 60 mg/m² or greater, 100 mg/m² or greater, 140 mg/m² or greater, or 180 mg/m² or greater. In a further embodiment, the amount of platinum compound in the lipid-based platinum compound formulation is 100 mg/m or greater, and the lipid-based platinum compound formulation is administered to the patient at least once every three weeks.

In another embodiment, the present invention relates to the aforementioned method, wherein the lipid-based platinum compound is prepared by (a) combining a platinum compound and a hydrophobic matrix carrying system; (b) establishing the mixture at a first temperature; (c) thereafter establishing the mixture at a second temperature, wherein the second temperature is cooler than the first temperature; and wherein the steps (b) and (c) are effective to increase the encapsulation of platinum compound. In a further embodiment the first temperature is from about 4° C. to about 70° C. In a further embodiment the second temperature is from about −25° C. to about 25° C. In a further embodiment the steps b) and c) are maintained for about 5 to 300 minutes.

These embodiments of the present invention, other embodiments, and their features and characteristics, will be apparent from the description, drawings and claims that follow.

### BRIEF DESCRIPTION OF THE DRAWINGS

FIG. 1 depicts the large decrease in toxicity of ip administration of lipid-based cisplatin (L-CDDP-ip) as compared to ip administration of cisplatin (CDDP-ip).

FIG. 2 depicts the increased amount of cisplatin in the blood stream from lipid-based cisplatin when administered intraperitoneally and intravenously as compared to free cisplatin administered intraperitoneally.

FIG. 3 depicts the increased amount of cisplatin in the blood stream from lipid-based cisplatin when administered intraperitoneally as compared to free cisplatin administered intraperitoneally.

FIG. 4 depicts the increased amount of cisplatin from lipid-based cisplatin in the kidney as compared to free cisplatin administered intraperitoneally.

FIG. 5 depicts the higher amount of cisplatin from lipid-based cisplatin in the liver as compared to free cisplatin when administered intraperitoneally.

FIG. 6 depicts the higher amount of cisplatin from lipid-based cisplatin in the lung as compared to free cisplatin when administered intraperitoneally.

FIG. 7 depicts the increased amount of cisplatin in lipid-based cisplatin in the spleen when administered intraperitoneally as compared to free cisplatin administered intraperitoneally.

FIG. 8 depicts the blood/kidney concentration ratio of platinum from lipid-based cisplatin and free cisplatin administered intraperitoneally.

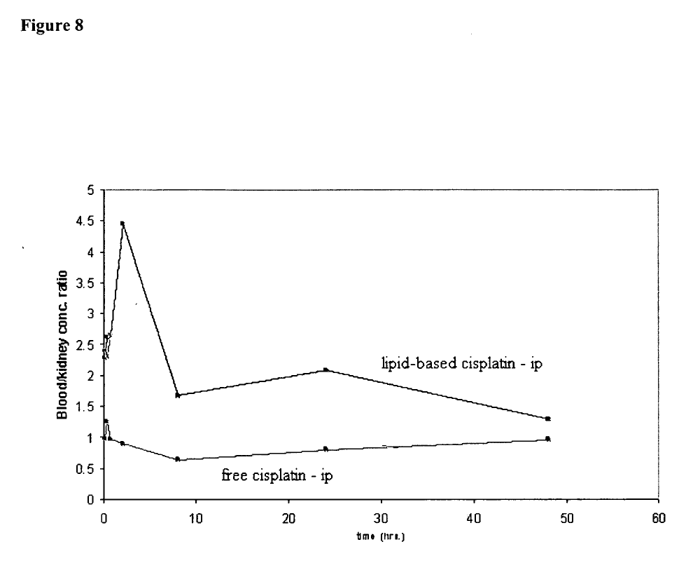

FIG. 9 depicts the increase in blood urea nitrogen (BUN) levels when free cisplatin is delivered either intravenously or intraperitoneally compared to lipid-based cisplatin administered by either method.

FIG. 10 depicts the survivial rate for mice with implanted viable human ovarian cancer cells line SK-OV₃-ip1 after ip administration of free cisplatin and lipid-based cisplatin.

FIG. 11 depicts the survival rate for mice with implanted viable L1210 tumor cells after ip administration of lipid-based cisplatin, non-cyclic temperature cisplatin liposomes, and soluble cisplatin.

### DETAILED DESCRIPTION OF THE INVENTION

I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “bioavailable” is art-recognized and refers to a form of the subject invention that allows for it, or a portion of the amount administered, to be absorbed by, incorporated to, or otherwise physiologically available to a subject or patient to whom it is administered.

The term “cancer treating effective amount” as used herein refers to the amount of lipid-based platinum compound formulation effective for the treatment of cancer. In one embodiment the cancer treating effective amount of lipid-based platinum compound formulation is typically about 100 mg/m² for ip delivery in a human.

The term “CDDP” stands for cis diamminedichloroplatinum, which is used interchangeably herein with “cisplatin”.

The terms “comprise” and “comprising” are used in the inclusive, open sense, meaning that additional elements may be included.

The term “hydrophobic matrix carrying system” is a lipid/solvent mixture prepared during the solvent infusion process described below.

The term “including” is used herein to mean “including but not limited to”. “Including” and “including but not limited to” are used interchangeably.

The term “intraperitoneal” or “intraperitoneally” or “ip” as used herein refers to administration of a therapeutic agent, such as, for example, an antineoplastic compound, such as a platinum compound, to the peritoneal cavity of a patient. The term “peritoneal cavity” as used herein refers to the serous membrane lining the abdominopelvic walls and investing the viscera.

The term “L-CDDP” stands for a lipid-based formulation of cis diamminedichloroplatinum which is used interchangeably herein with “lipid-based cisplatin”.

The terms “lipid-based platinum compound” as used herein refers to a composition comprising a lipid and a platinum compound. In some embodiments the lipid-based platinum compound can be in the form of a liposome. In some embodiments the ratio of platinum compound to lipid in the lipid-based platinum compound can be between about 1:5 by weight and 1:50 by weight. In a further embodiment, the ratio of platinum compound to lipid in the lipid-based platinum compound can be between about 1:5 and about 1:30. In a further embodiment, the ratio of platinum compound to lipid in the lipid-based platinum compound can be between about 1:5 by weight and 1:25 by weight. In still other embodiments, the platinum compound can be cisplatin.

The term “mammal” is known in the art, and exemplary mammals include humans, primates, bovines, porcines, canines, felines, and rodents (e.g., mice and rats).

A “patient,” “subject” or “host” to be treated by the subject method may mean either a human or non-human animal.

The term “pharmaceutically-acceptable salts” is art-recognized and refers to the relatively non-toxic, inorganic and organic acid addition salts of compounds, including, for example, those contained in compositions of the present invention.

The term “solvent infusion” is a process that includes dissolving one or more lipids in a small, preferably minimal, amount of a process compatible solvent to form a lipid suspension or solution (preferably a solution) and then adding the solution to an aqueous medium containing bioactive agents. Typically a process compatible solvent is one that can be washed away in a aqueous process such as dialysis. The composition that is cool/warm cycled is preferably formed by solvent infusion. Alcohols are preferred as solvents, with ethanol being a preferred alcohol.

“Ethanol infusion,” is a type of solvent infusion that includes dissolving one or more lipids in a small, preferably minimal, amount of ethanol to form a lipid solution and then adding the solution to an aqueous medium containing bioactive agents. A “small” amount of solvent is an amount compatible with forming liposomes or lipid complexes in the infusion process.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic index” is an art-recognized term which refers to the ratio of a quantitative assessment of toxicity to a quantitative assessment of efficacy of a drug, e.g. LD₅₀/ED₅₀ in the case of animals. The term “LD₅₀” is art recognized and refers to the amount of a given toxic substance that will elicit a lethal response in 50% of the test organisms. This is sometimes also referred to as the median lethal dose. The term “ED₅₀” is art recognized and refers to the median effective dose.

The term “treating” is art-recognized and refers to curing as well as ameliorating at least one symptom of any condition or disease.

II. Lipids

The lipids used in forming the liposomes for ip or iv delivery of an antineoplastic agent may be synthetic, semi-synthetic or naturally-occurring lipids, including phospholipids, tocopherols, sterols, fatty acids, glycoproteins such as albumin, negatively-charged lipids and cationic lipids. In terms of phosholipids, they could include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC. Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (PIs) and the phosphatidyl serines (PSs). Examples include DMPG, DPPG, DSPG, DMPA, DPPA, DSPA, DMPI, DPPI, DSPI, DMPS, DPPS and DSPS.

III. Liposomes

Liposomes are completely closed lipid bilayer membranes containing an entrapped aqueous volume. Liposomes used for the parenteral delivery of an antineoplastic compound may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoffet al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

Another method of preparing liposomes is the “solvent infusion” process. Solvent infusion is a process that includes dissolving one or more lipids in a small, preferably minimal, amount of a process compatible solvent to form a lipid suspension or solution (preferably a solution) and then adding the solution to an aqueous medium containing, for example, platinum compounds. Typically a process compatible solvent is one that can be washed away in an aqueous process such as dialysis. The composition that is cool/warm cycled is preferably formed by solvent infusion, with ethanol infusion being preferred.

The process for producing lipid-based platinum compound formulations may comprise mixing a platinum compound with an appropriate hydrophobic matrix and subjecting the mixture to one or more cycles of two separate temperatures. The process is believed to form active platinum compound associations.

In aqueous solution, when the platinum compound is cisplatin, it may form large insoluble aggregates with a diameter of greater than a few microns. In the presence of a amphipathic matrix system, such as a lipid bilayer, cisplatin-lipid associations form. For example, the associations may be formed in the internal aqueous space, the hydrocarbon core region of a lipid bilayer, or the liposome interface or headgroup. During the warming cycle of the process, it is believed that cisplatin is returned to solution at a greater rate in aqueous regions of the process mixture than from the lipid-matrix. As a result of applying more than one cool/warm cycle, cisplatin accumulates further from the lipid-matrix. Without limiting the invention to the proposed theory, experimentation indicates that the cisplatin-lipid associations cause the immediate surroundings of the interfacial bilayer region to be more hydrophobic and compact. This results in a high level of entrapment of active platinum compound as cooling and warming cycles are repeated.

The process comprises combining the platinum compound with a hydrophobic matrix carrying system and cycling the solution between a warmer and a cooler temperature. Preferably the cycling is performed more than one time. More preferably the step is performed two or more times, or three or more times. The cooler temperature portion of cycle can, for example, use a temperature from about −25° C. to about 25° C. More preferably the step uses a temperature from about −5° C. to about 25° C. or from about 1° C. to about 20° C. For manufacturing convenience, and to be sure the desired temperature is established, the cooler and warmer steps can be maintained for a period of time, such as approximately from 5 to 300 minutes or 30 to 60 minutes. The step of warming comprises warming the reaction vessel to from about 4° C. to about 70° C. More preferably the step of warming comprises heating the reaction vessel to about 45° C. or to about 55° C. The above temperature ranges are particularly preferred for use with lipid compositions comprising predominantly diphosphatidycholine (DPPC) and cholesterol.

Another way to consider the temperature cycling is in terms of the temperature differential between the warmer and the cooler steps of the cycle. This temperature differential can be, for example, about 25° C. or more, such as a differential from about 25° C. to about 70° C., preferably a differential from about 40° C. to about 55° C. The temperatures of the cooler and higher temperature steps are selected on the basis of increasing entrapment of active platinum compound. Without being limited to theory, it is believed that it is useful to select an upper temperature effective substantially increase the solubility of active platinum compound in the processed mixture. Preferably, the warm step temperature is about 50° C. or higher. The temperatures can also be selected to be below and above the transition temperature for a lipid in the lipid composition.

The temperatures appropriate for the method may, in some cases, vary with the lipid composition used in the method, as can be determined by ordinary experimentation.

The platinum compound to lipid ratio seen in the lipid-based platinum formulations used in the present invention may be between about 1:5 by weight and about 1:50 by weight. More preferably the platinum compound to lipid ratio achieved is between about 1:5 by weight and about 1:30 by weight. Most preferably the platinum compound to lipid ratio achieved is between about 1:5 by weight and about 1:25 by weight.

The liposomes have a mean diameter of approximately 0.01 microns to approximately 3.0 microns, preferably in the range about 0.1 to 1.0 microns. More preferably, the mean diameter is from about 0.2 to 0.5 microns. The sustained release property of the liposomal product can be regulated by the nature of the lipid membrane and by inclusion of other excipients (e.g., sterols) in the composition.

In a preferred embodiment of the invention the liposome contains about 50 to about 100 mol % DPPC and about 0 to about 50 mol % cholesterol. More preferably, the liposome contains about 50 to about 65 mol % DPPC and about 35 to about 50 mol % cholesterol.

Liposomes can also be prepared by the methods disclosed in copending U.S. patent application Ser. No. 10/383,004, filed Mar. 5, 2003; Ser. No. 10/634,144, filed Aug. 4, 2003; Ser. No. 10/224,293, filed Aug. 20, 2002; and Ser. No. 10/696,389, filed Oct. 29, 2003, the specifications of which are incorporated herein in their entirety.

IV. Platinum Compounds

The platinum compounds that may be used in the present invention include any compound that exhibits the property of preventing the development, maturation, or spread of neoplastic cells. Non-limiting examples of platinum compounds include cisplatin, carboplatin (diammine(1,1-cyclobutanedicarboxylato)-platinum(II)), tetraplatin (ormaplatin) (tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine) platinum(IV)), JM335 (trans-amminedichloro(cyclohexylamine)dihydroxoplatinum(IV)), and (trans, trans, trans)bis-mu-(hexane-1,6-diamine)-mu-[diamine-platinum(II)]bis[diamine(chloro) platinum(II)]tetrachloride. In another embodiment the platinum compound is cisplatin. Depending on the environment, cisplatin may exist in a cationic aquated form wherein the two negatively charged chloride atoms have been displaced by two neutral water molecules. Because the aquated form of cisplatin is cationic, anionic lipids such as glycerols help to stabilize the lipid-based formulation, but may also hinder release on the cisplatin. The non-aquated, neutral form of cisplatin is harder to stabilize but has different release kinetics. It is considered an advantage of the present invention that in certain embodiments the lipid-based cisplatin formulations comprise neutral cisplatin and neutral lipids. Because of the equilibrium between neutral, non-aquated cisplatin and cationic, aquated cisplatin, one may favor neutral, non-aquated cisplatin by preparing a formulation with a low pH and high NaCl concentration. In this embodiment a substantial amount of the cationic, aquated form of cisplatin would not form until the neutral, non-aquated cisplatin was delivered into the interior of a cell.

In other embodiments, other therapeutic agents may be used with the platinum compounds. The other therapeutic agents may have antineoplastic properties. Non-limiting examples of antineoplastic compounds include altretamine, amethopterin, amrubicin, annamycin, arsenic trioxide, asparaginase, BCG, benzylguanine, bisantrene, bleomycin sulfate, busulfan carmustine, cachectin, chlorabucil, 2-chlorodeoxyadenosine, cyclophosphamide, cytosine arabinoside, dacarbazine imidazole carboxamide, dactinomycin, daunomycin, 3′-deamino-3′-morpholino-13-deoxo-10-hydroxycarminomycin, 4-demethoxy-3-deamino-3-aziridinyl-4 -methylsulphonyl-daunorubicin, dexifosfamide, dexamethasone, diarizidinylspermine, dibromodulcitol, dibrospidium chloride, 1-(11-dodecylamino-10-hydroxyundecyl)-3,7-dimethylxanthine, doxorubicin, elinafide, epipodophyllotoxin, estramustine, floxuridine, fluorouracil, fluoxymestero, flutamide, fludarabine, fotemustine, galarubicin, glufosfamide, goserelin, GPX100, hydroxyurea, idarubicin HCL, ifosfamide, improsulfan tosilate, isophosphamide, interferon alfa, interferon alfa 2a, interferon alfa 2b, interferon alfa n3, interferon gamma, interleukin 2, irinotecan, irofulven, leucovorin calcium, leuprolide, levamisole, lomustine, megestrol, L-phenylalanie mustard, L-sarcolysin, melphalan hydrochloride, mechlorethamine, MEN10755, mercaptopurine, MESNA, methylprednisolone, methotrexate, mitomycin, mitomycin-C, mitoxantrone, nimustine, paclitaxel, pinafide, pirarubicin, plicamycin, prednimustine, prednisone, procarbazine, profiromycin, pumitepa, ranimuistine, sertenef, streptozocin, streptozotocin, tamoxifen, tasonermin, temozolomide, 6-thioguanine, thiotepa, tirapazimine, triethylene thiophosporamide, trofosfamide, tumor necrosis factor, valrubicin, vinblastine, vincristine, vinorelbine tartrate, and zorubicin.

Also included as suitable platinum compounds used in the methods of the present invention are pharmaceutically acceptable addition salts and complexes of platinum compounds. In cases wherein the compounds may have one or more chiral centers, unless specified, the present invention comprises each unique racemic compound, as well as each unique nonracemic compound.

In cases in which the platinum compounds have unsaturated carbon-carbon double bonds, both the cis (Z) and trans (E) isomers are within the scope of this invention. In cases wherein the neoplastic compounds may exist in tautomeric forms, such as keto-enol tautomers, such as 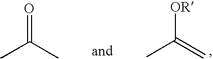
each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included as suitable platinum compounds used in the methods of the present invention are prodrugs of the platinum compounds. Prodrugs are considered to be any covalently bonded carriers which release the active parent compound in vivo.

The present invention, in part, discloses methods of treating cancer more effectively which may have lower nephrotoxicity previously not disclosed. By using lipid-based formulations and ip delivery, a more potent and efficient cancer treatment is achieved.

V. Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 50 mg per kg.

Dosage amounts are also commonly administered as mg/m² which stands for milligrams of drug (e.g. platinum compound) per body surface area. Generally, dosage amounts for platinum compounds may be about 60 mg/m or greater, 100 mg/m² or greater, 140 mg/m² or greater, or 180 mg/m² or greater. Dosage amounts of about 140 mg/m² or greater are generally considered at the high end of tolerance, but an advantage of the present invention is that the platinum compound is administered as part of a lipid-based formulation which decreases the sub-acute toxicities of the platinum compound. It is therefore envisioned by the inventors that higher than normal dosage amounts of platinum compound may be administered to the patient without unwanted toxic side effects. Higher dosages may lead to longer duration cycles between dosages and greater convenience for the patient. For example, dosage amounts are generally administered to the patient once about every three weeks. If higher dosage amounts of platinum compound can be administered safely to the patient then the cycle time may be increased to once about every four, five, six, seven, or even eight weeks. Longer cycle times means less trips to a care facility for treatment and less times the patient would have to undergo the administration process.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the antineoplastic compound) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

VI. Pharmaceutical Formulation

The pharmaceutical formulation of the antineoplastic compound may be comprised of an aqueous dispersion of liposomes. The formulation may contain lipid excipients to form the liposomes, and salts/buffers to provide the appropriate osmolarity and pH. The pharmaceutical excipient may be a liquid, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### EXEMPLIFICATION

### Example 1

Reduction of sub-acute toxicity of cisplatin by iv or ip administration when administered as a lipid-based formulation. ICR mice, male and female, 6-7 weeks old, were divided into 24 groups with 10 mice in each. Five mice were housed in each cage with free access to standard mouse food and water. Each group of mice was injected with lipid-based cisplatin formulations prepared according to the following. The lipid-based cisplatin formulation used here contained 1 mg/ml cisplatin, 16 mg/ml DPPC, and 7.9 mg/ml cholesterol in 0.9% NaCl solution. An aliquot (50%) of the sample was treated by 3 cycles of cooling to 4° C. and warming to 50° C. The aliquot, in a test tube, was cooled by refrigeration, and heated in a water bath. The resulting unentrapped cisplatin (free cisplatin) was washed away by dialysis. The lipid-based cisplatin in the form of liposomes were injected through iv (tail vein) or ip route. The liposomes had a mean diameter of about 0.39 μm. The formulations, doses, and administration routes are listed in Table 1.

TABLE 1
Dose and administration route for lipid-based cisplatin
sub-acute toxicity study.

| Formulation | Route | Dose (mg Cisplatin/kg mouse) | | | | | | |
|---|---|---|---|---|---|---|---|---|
| Lipid-Based | ip | 23 | 27 | 31 | 35 | 40 | 45 | 50 |
| Cisplatin | | | | | | | | |
| Cisplatin | ip | 9 | 12 | 15 | 18 | 21 | 24 | 27 |

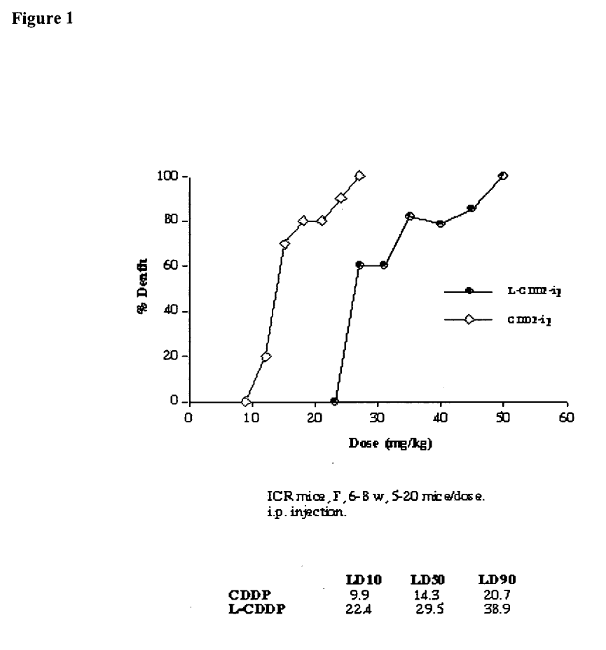

Starting one week before the administration, body weights of the mice were measured every two days until the end of the experiment. The animals were observed daily and the death was recorded. A curve of percent survival verses time (days) post administration for each formulation with each injection route was calculated (FIG. 1). The LD₁₀, LD₅₀ and LD₉₀ of each formulation under each injection route were estimated. The computer fitted results are listed in Table 2.

TABLE 2
Lethal toxicity of lipid-based cisplatin and
cisplatin after ip and iv injection.

| | | Lethal Dose (mg Cisplatin/kg mouse) | | |
|---|---|---|---|---|
| Formulation | Route | LD₁₀ | LD₅₀ | LD₉₀ |
| Lipid-Based | ip | 22.4 | 29.5 | 38.9 |
| Cisplatin | | | | |
| Cisplatin | ip | 9.9 | 14.3 | 20.7 |

The result indicate that the sub-acute toxicity of ip lipid-based cisplatin was 2-fold lower than ip cisplatin, whereas not nearly as great of change was observed for iv lipid-based cisplatin.

### Example 2

Pharmacokinetics and organ distribution in animals of ip and iv injected lipid-based cisplatin and cisplatin (Part I). The mice (the same as from Example 1) were divided into 4 groups with 24 mice in each. They were injected with ip lipid-based cisplatin (12 mg/kg), ip cisplatin (12 mg/kg), iv lipid-based cisplatin (8 mg/kg), and iv cisplatin (8 mg/kg), separately. The lipid-based cisplatin formulation were prepared in the same manner as in Example 1. At each designed time point, e.g., 2-5 min, 20 min, 40 min, 2 h, 8 h, 1 day, 2 days, and 3 or 5 days after injection, 3 mice from each group were anesthetized by ip injection of 35-50 mg/kg of Nembutal, then the blood was drown and heart, kidney, liver, lung, small intestine, and spleen were resected and homogenized after adding 4-fold pure water. The Platinum concentration in each sample was determined with AA method. The content of Pt (μg of Pt in 1 ml of blood or 1 gram of tissue) was calculated and used for presenting the kinetic characteristics of each formulation under two different administration routes.

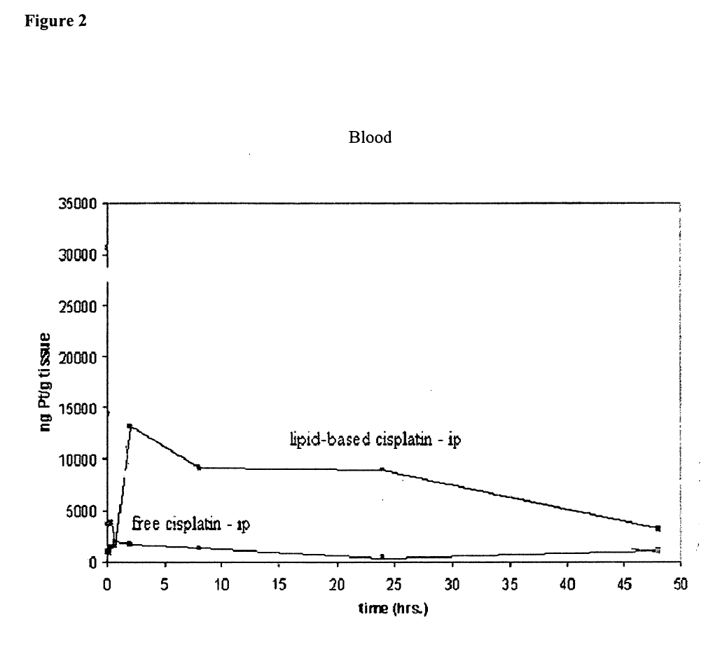

The results indicated that in the blood, the Cmax and AUC of lipid-based cisplatin was 3- and 6-fold higher than that of cisplatin, respectively (FIG. 2).

### Example 3

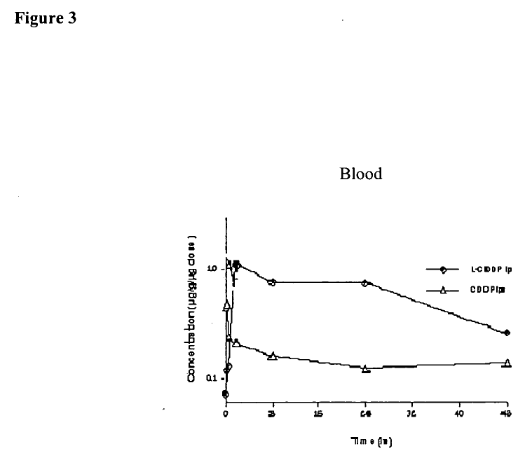

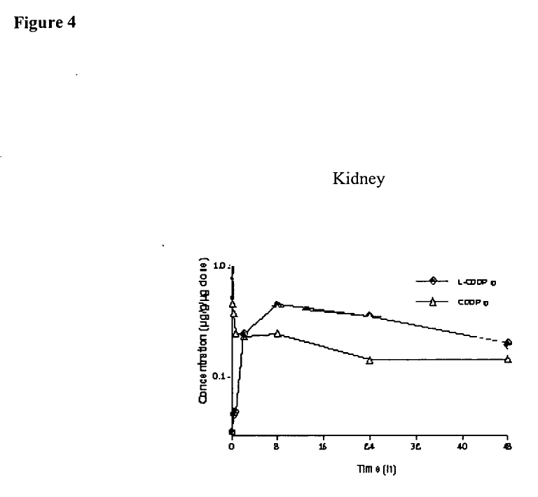

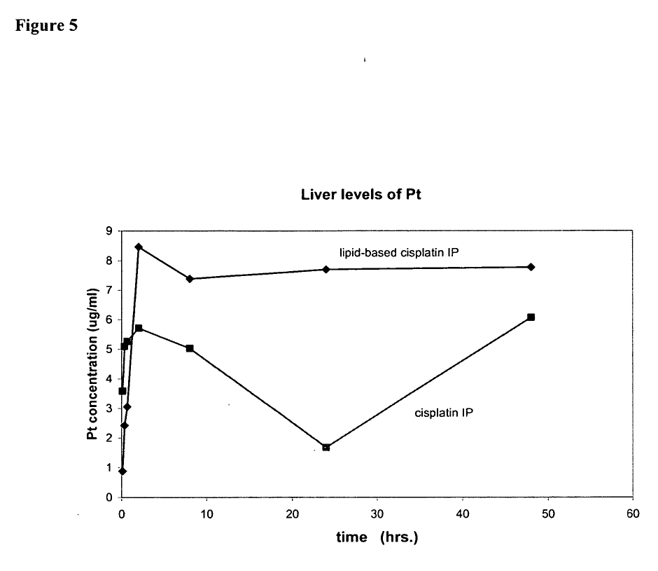

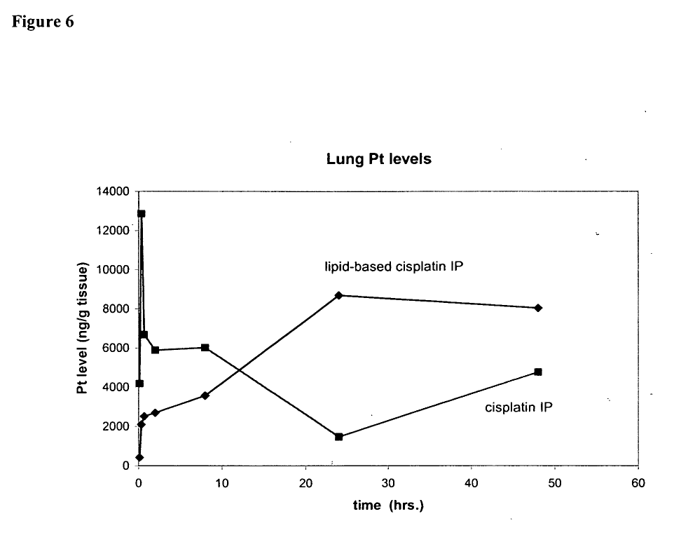

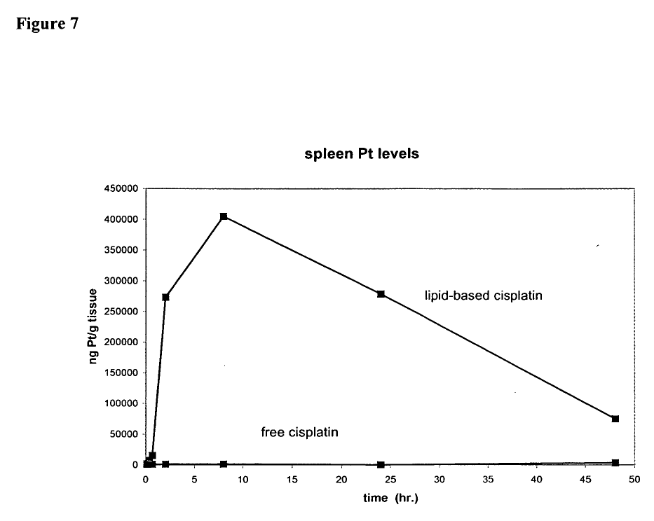

Pharmacokinetics and organ distribution in animals of ip and iv injected lipid-based cisplatin and cisplatin (Part II). Sixty ICR mice (female, 7 weeks old) were divided into 4 groups. They received intraperitoneal or intravenous injection of L-CDDP or CDDP, separately. The dose was 12 mg/kg for ip L-CDDP and 8 mg/kg for the rest of treatment groups. At each designed time point, three to four mice were anaesthetized with 70 mg/kg of Nembutal ip (e.g., 3, 20, and 40 min, and 2, 8, 24, 48, and 72 h). The blood was drawn from the inferior vena cava. Organs including duodenum, kidney, liver, lung, and spleen were resected from the mice. The blood and organ samples were homogenized in distilled water (4-fold of the sample weight) and digested with nitric acid. The platinum concentration in each sample was measured by Inductively Coupled Plasma-Mass Spectrometer (ICP-MS). The pharmacokinetics profiles (FIGS. 3-7, all Y-axes are concentration of μg platinum in one gram of tissue or fluid per mg of injected dose) and parameters (Tables 3 and 4) of each formulation were simulated and calculated. It was found that 1) uptake of platinum in the spleen was significantly enhanced by the liposomal formulation irrespective of the injection routes, the AUCs and Cₘₐₓ were 26˜47 -fold higher than those of CDDP, p<0.0003; 2) with ip injection, the circulation AUC and elimination t_{1/2} of L-CDDP were 4- and 15-fold (p<0.006) higher than those of CDDP, but this phenomenon was not found after iv injection; 3) the AUC and t_{1/2} of ip L-CDDP were also higher than those of iv L-CDDP (ip vs iv: AUC, 1.9-fold, p=0.04; t_{1/2}, 5.7-fold, p=0.006); 4) the lung and liver platinum uptake of iv L-CDDP were slightly higher than that of iv CDDP (Lung: p=0.043; Liver: p=0.051); 5) the kidney platinum uptake of L-CDDP was higher than that of CDDP (p=0.046). These results imply that the formulation and administration route both play important role on the PK and organ distribution of the drug, and ip L-CDDP showed sustained release function.

TABLE 3
Circulation AUC and t_{1/2}.

| | AUC (h μg/g/μg) | t_{1/2} (h) |
|---|---|---|
| | ip | ip |
| L-CDDP | 37.60^{a} | 26.58^{b} |
| CDDP | 7.08 | 1.82 |

^{a}p = 0.008 (L-CDDP vs CDDP);
^{b}p = 0.006 (L-CDDP vs CDDP) by two side Log rank test.

TABLE 4
Organ AUC and Cₘₐₓ.

| ip | |
|---|---|
| AUC (h μg/g/μg) | Cₘₐₓ (μg/g/μg) |

| | L-CDDP | CDDP | L-CDDP | CDDP |
|---|---|---|---|---|
| Duodenum | 5.49 | 8.32 | 0.30 | 0.45 |
| Kidney | 15.60^{c} | 8.61 | 0.46 | 0.46 |
| Liver | 29.99 | 28.99 | 0.71 | 0.71 |
| Lung | 26.90 | 29.00 | 0.73 | 1.61 |
| Spleen | 331.86^{e} | 10.45 | 11.32^{f} | 0.43 |
| Heart | 2.63 | 2.67 | 0.07 | 0.18 |

Two side Log rank test was used to evaluate the significance of the differences of L-CDDP versus CDDP in AUC or Cₘₐₓ values. Statistically significant pairs (p<0.05) were labeled with a superscript letter. Their p values are as follows: c, p=0.046; d, p=0.008; e, p=0.0002; f, p=0.0001; g, p=0.0003; h, p=0.0002.

### Example 4

Nephrotoxicity. ICR mice, 7 weeks old, female, were divided into 4 groups with 3 to 4 mice in each. They were injected with maximum tolerated dose (MTD) of L-CDDP or CDDP via iv or ip. Four days after the injection, the mice were euthanized with Nembutal ip. The blood was drawn and the serum was isolated. The blood urea nitrogen (BUN) was quantitatively measured with a colorimetric method at Antech Diagnostics. Organs including duodenum, heart, kidney, liver, lung, and spleen were resected from the mice and fixed with 10% buffered Formalin. The fixed tissues were processed with standard procedure for H and E staining. A pathology expert Dr. Carman Tornos at the Memorial Sloan-Kettering Cancer Center examined kidney tissues and gave a toxicity grade to each kidney tissue sample. The grading was based on the general pathology guidelines for kidney toxicity.

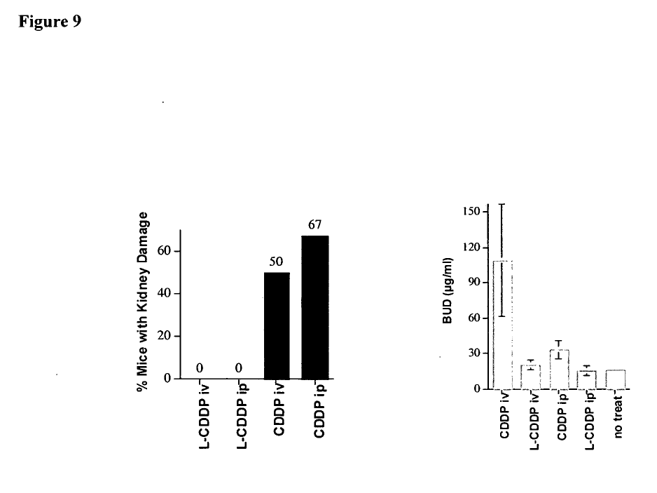

The pathological results demonstrate that irrespective of administration routes, CDDP caused severe nephrotoxicity in more than 50% mice receiving the treatment, but L-CDDP did not cause any nephrotoxicity. The similar conclusion can be drawn from BUN test (FIG. 9), where iv CDDP significantly increased BUN level by 6.8-fold compared with normal controls (p=0.008), and ip CDDP caused much less BUN accumulation, only 2.1-fold increase compared to normal controls (p=0.04), but L-CDDP injected by either route did not cause BUN level elevation.

### Example 5

Preclinical in vivo antitumor activity of lipid-based cisplatin in a Murine L1210 tumor model. The purpose of this experiment is to assess the in vivo antitumor activity of lipid-based cisplatin against a cavity confined tumor (ascitic L1210 leukemia) by local ip administration. Lipid-based cisplatin was compared to free cisplatin for viable L1210 tumor cells. The test articles and materials are presented below in Table 5. The lipid-based cisplatin was prepared in the same manner as in Example 1.

TABLE 5
Test articles and materials.

| | |
|---|---|
| Test Articles | Cisplatin in 0.9% saline |
| | 1.0 mg/ml cisplatin |
| | Lipid-Based Cisplatin |
| | 0.82 mg/ml cisplatin |
| Test Animals | B6D2F1/Hsd hybrid mice from Harlan |
| | 5 male/group except control (9 male), 7 groups |
| Tumor Cells | Viable L1210 tumor cells, |
| | One million per mouse, transplanted in vivo |

The procedure was as outlined below and summarized in Table 6:
1. Day 0, inoculate 6 groups of 5 male mice and 1 group of 9 male control animals with one million viable L1210 cells per mouse by ip injection.
2. Administer ascending doses of either cisplatin solution or lipid-based cisplatin to groups of 5 animals. Cisplatin solution: dose ip at 3.0 and 4.5 mg/Kg on days 3, 7, and 11. Each dose level represents one group of five mice. Lipid-based cisplatin: dose ip at 3.0, 4.5, 6.0 and 9.0 mg/Kg on days 3, 7, and 11. Control group contains 9 untreated mice.

3. Mice were monitored daily for deaths and or signs of clinical illness. The date of euthanasia was recorded for the purpose of experimental end-points. A total of 39 mice divided into 7 groups were studied. At the end point survival was assessed and expressed as % T/C (percent median survival of treated group: median survival of control group.)

TABLE 6
Procedural parameters

| Group | Mice | Drug tested | ip Dose | Day 0 | Day 3 | Day 7 | Day 11 |
|---|---|---|---|---|---|---|---|
| 1 | 9 male | no treatment | control | inoculate | | | |
| 2 | 5 male | free cisplatin | 3 mg/kg | inoculate | 1 mg/kg | 1 mg/kg | 1 mg/kg |
| 3 | 5 male | free cisplatin | 4.5 mg/kg | inoculate | 1.5 mg/kg | 1.5 mg/kg | 1.5 mg/kg |
| 4 | 5 male | Lipid-based | 3 mg/kg | inoculate | 1 mg/kg | 1 mg/kg | 1 mg/kg |
| | | cisplatin | | | | | |
| 5 | 5 male | Lipid-based | 4.5 mg/kg | inoculate | 1.5 mg/kg | 1.5 mg/kg | 1.5 mg/kg |
| | | cisplatin | | | | | |
| 6 | 5 male | Lipid-based | 6 mg/kg | inoculate | 2 mg/kg | 2 mg/kg | 2 mg/kg |
| | | cisplatin | | | | | |
| 7 | 5 male | Lipid-based | 9 mg/kg | inoculate | 3 mg/kg | 3 mg/kg | 3 mg/kg |
| | | cisplatin | | | | | |

The results from the experiment are summarized in Table 7.

TABLE 7
Survival data as measured by % T/C.

| Group | Drug tested | Dose | Median survival* | % T/C |
|---|---|---|---|---|
| 1 | no treatment | Control | 13.5 | 100 |
| 2 | free cisplatin | 3 mg/kg | 26.5 | 196 |
| 3 | free cisplatin | 4.5 mg/kg | 29.5 | 218 |
| 4 | Lipid-based | 3 mg/kg | 19.5 | 144 |
| | cisplatin | | | |
| 5 | Lipid-based | 4.5 mg/kg | 23.5 | 174 |
| | cisplatin | | | |
| 6 | Lipid-based | 6 mg/kg | 25.5 | 189 |
| | cisplatin | | | |
| 7 | Lipid-based | 9 mg/kg | 25.5 | 189 |
| | cisplatin | | | |

*Median survival is defined as day of death for 50% of mice with each group.

Day of death for 50% of mice within each group was determined and an initial % Treated/Control (T/C) value was recorded in the above table. At the optimal dose, the cisplatin in lipid-based cisplatin does not lose any of its antitumor activity compared to free cisplatin.

### Example 6

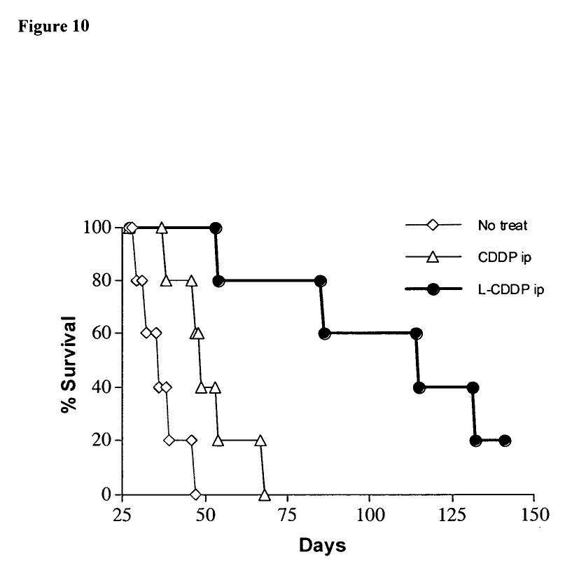

Antitumor activity of L-CDDP against human ovarian cancer xenograft. Nude mice, female, 6-7 weeks old, were intraperitoneally inoculated with human ovarian cancer cell line SK-OV₃-ip1 (1.5×10⁶ cells/mouse). One week after the inoculation, the mice were randomly divided into 3 groups with 5 mice in each. One group of mice was given single bolus ip injection of CDDP with MTD (9 mg/kg) to mimic the current chemotherapy (positive control). Another group was treated with single bolus ip injection of L-CDDP with MTD (23 mg/kg). The third group of mice without treatment was used as negative control. The mice were observed on a daily basis. Death of mice was recorded and the increased lifespan (ILS) was calculated. Results are presented in FIG. 10.

### Example 7

Comparison of lipid-based cisplatin prepared by the cyclic temperature effusion process and non cyclic temperature cisplatin liposomes. The lipid-based cisplatin prepared by the cyclic temperature effusion process were prepared as in Example 1 and contained 1.1 mg/ml cisplatin and 27 mg/ml total lipid. The non cyclic temperature cisplatin liposomes were prepared according to the following procedure.
1. DPPC (3.0 g) and cholesterol (1.2 g) were co-dissolved in 20 mL of ethanol.
2. Cisplatin (200 mg) was dissolved in 0.9% saline (200 ml).
3. The lipid/ethanol solution was infused into the cisplatin solution as it was being well-stirred (liposomes formed).
4. The lipid-cisplatin suspension was dialyzed to wash away un-entrapped cisplatin.
5. The resulting liposomal cisplatin contained 0.03 mg/ml total cisplatin (75% of total cisplatin was entrapped and 25% was un-entrapped); the total lipid concentration was 21 mg/ml.

The mice were given equivalent amounts of cisplatin containing therapeutics based on the amount of lipid instead of the amount of cisplatin. This was necessary because in non cyclic temperature cisplatin liposomes the lipid to cisplatin ratio is so high that it is not possible to administer that much lipid necessary to equal the amount of cisplatin in the lipid-based formulations prepared as in Example 1.

Female DBA/2 mice (Charles Rivers) were used. Thirty (30) mice were injected with 2×10⁶ L1210 cells ip on Day 0. On day 1, the mice were weighed and randomized into 3 groups of 10 mice. On days 5, mice received a single bolus intraperitoneal injection of soluble cisplatin (6 mg/kg), lipid-based cisplatin (6 mg/kg, ip) or non cyclic temperature cisplatin liposomes (equal lipid to lipid-based cisplatin, 0.2 mg/kg). Survival was monitored. Mice were weighed daily after day 10. Mice that lost 20% or greater of their starting weight were euthanized by CO₂ inhalation. The date of their death was recorded on data sheets. Median survival was calculated by Prism GraphPad.

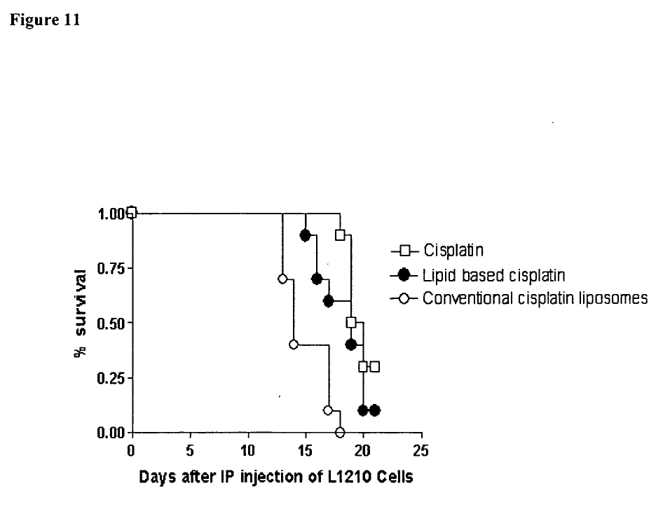

The results of experiments where both types of cisplatin formulations were administered intraperitoneally to mice with implanted viable L1210 tumor cells are depicted in FIG. 11. There was no significant difference between survival curves of mice that received lipid-based cisplatin intraperitoneally and those that received soluble cisplatin intraperitoneally (p=0.20). All survival curves of cisplatin-treated groups were significantly different from the mice that received non cyclic temperature cisplatin liposomes (p=0.0035, and p<0.0001, respectively). The days of median survival were 19 for lipid-based cisplatin, ip; 19.5 for free cisplatin, ip; and 14 for non cyclic temperature cisplatin liposomes, ip.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of treating cancer in a patient comprising administering to the patient intraperitoneally, a cancer treating effective amount of a lipid-based platinum compound formulation.

2. The method of claim 1, wherein the platinum compound in the lipid-based platinum compound formulation is administered at a concentration of 1 mg/ml.

3. The method of claim 1, wherein the platinum compound is selected from the group consisting of: cisplatin, carboplatin (diammine(1,1-cyclobutanedicarboxylato)-platinum(II)), tetraplatin (ormaplatin) (tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine) platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

4. The method of claim 1, wherein the platinum compound is cisplatin.

5. The method of claim 1, wherein the lipid is comprised of a member selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic aicd (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic aicd (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

6. The method of claim 1, wherein the lipid is a mixture of a phospholipid and a sterol.

7. The method of claim 1, wherein the lipid is a mixture of DPPC and cholesterol.

8. The method of claim 1, wherein the lipid is a mixture of DPPC from 50 to 65 mol % and cholesterol from 35 to 50 mol %.

9. The method of claim 1, wherein the cancer is selected from the following: melanoma, testis (germ cell), osteosarcoma, soft tissue sarcoma, thyroid cancer, colon cancer, ovarian cancer, cancer of the kidney, breast cancer, colorectal cancer, prostate cancer, bladder cancer, uterine cancer, lung cancer, stomach cancer, liver cancer, endometrial, or squamous cell carcinomas of the head and neck

10. The method of claim 1, wherein the cancer is ovarian cancer.

11. The method of claim 1, wherein the cancer is colon cancer.

12. The method of claim 1, wherein the ratio of platinum compound to lipid in the lipid-based platinum compound formulation is between 1:5 by weight and 1:50 by weight.

13. The method of claim 1, wherein the lipid-based platinum compound formulation comprises liposomes having a mean diameter of 0.01 microns to 3.0 microns.

14. The method of claim 1, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

15. The method of claim 1, wherein the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

16. The method of claim 1, wherein the lipid is a mixture of DPPC and cholesterol in a 2 to 1 ratio by weight, the ratio of platinum compound to lipid in the lipid-based platinum compound formulation is about 1:20 by weight, the lipid-based platinum compound formulation comprises liposomes having a mean diameter of about 0.40 microns, and wherein the platinum compound is cisplatin.

17. The method of claim 1, wherein the patient is a human.

18. The method of claim 1, wherein the lipid-based platinum compound formulation is administered to the patient at least once every three weeks.

19. The method of claim 1, wherein the amount of platinum compound in the lipid-based platinum compound formulation is 60 mg/m² or greater, 100 mg/m² or greater, 140 mg/m² or greater, or 180 mg/m² or greater.

20. The method of claim 1, wherein the amount of platinum compound in the lipid-based platinum compound formulation is 100 mg/m² or greater, and the lipid-based platinum compound formulation is administered to the patient at least once every three weeks.
